(19) United States
(12) Patent Application Publication
(10) Pub. No.: US 2008/0218732 A1
(43) Pub. Date: Sep. 11, 2008

(54) Infrared Scanner for Biological Applications

(75) Inventors: Samson Mil'shtein, Chelmsford, MA (US); Niyom Lue, Nahant, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: University of Massachusetts Lowell, Lowell, MA (US)

(21) Appl. No.: 11/996,103

(22) PCT Filed: Jul. 24, 2006
(86) PCT No.: PCT/US06/28719

§ 371 Date: Jan. 18, 2008

Related U.S. Application Data
(60) Provisional application No. 60/703,030, filed on Jul. 27, 2005.

(51) Int. Cl. A61B 6/00

(52) U.S. Cl. 356/51

(57) ABSTRACT

Methods and systems for generating near-infrared (NIR) images of biological targets are discussed. In one aspect, one or more radiation sources illuminate a target, with one or more detectors receiving the transmitted radiation. Such equipment can be used to generate a plurality of NIR images of a target. The images can be converted into frequency space, combined using chosen weighting factors, and deconvoluted into the spatial domain to provide a composite image. The composite image can have enhanced quality relative to the individual images, allowing for a richer set of information to be displayed. Other aspects such as scanning, background illumination correction, the use of filters, and additional techniques are also discussed.

### BACKGROUND OF THE INVENTION

The present invention is generally directed to methods and systems for generating infrared images of biological objects, and more particularly, to such systems and methods that provide near infrared (NIR) images of biological objects in which not only blood vessels but also other objects and tissue types, e.g., tendons and bones, ligaments, blood vessels, tumors, and cartilages can be visible.

X-ray imaging systems are frequently used in many medical facilities today. Though x-ray imaging can be useful for diagnosing a variety of medical conditions, it has limitations. Since x-ray attenuation depends upon the effective atomic density of a scanned object, such imaging systems cannot provide adequate differentiation of biological structures in soft tissue regions. Though techniques such as administration of particular liquids to a subject may increase the contrast of a subsequently obtained image, such enhancement methods are invasive and inconvenient for the patient. Furthermore, obtaining real-time x-ray images of moving structures (e.g., a patient's fingers) is impractical, e.g., as a result of imaging artifacts.

A variety of systems for infrared imaging of biological objects are also known. An example of such systems is an oxymeter that utilizes the differential absorption of infrared radiation by oxygenated and non-oxygenated hemoglobin present in blood vessels for visualization of those vessels. Other applications include, e.g., in-situ examination of tissue during surgery and examination of naturally densified tissue at the sites of tumors.

The conventional infrared imaging systems, however, suffer from a number of shortcomings. For example, the infrared wavelengths utilized by many of these systems do not penetrate deep into the tissue, and hence can only be detected in the reflection mode. Even those systems that operate in the transmission mode (i.e., collect a portion of radiation transmitted though an object to be imaged) allow visualization of only the object's soft tissue.

Thus, there is a need for enhanced imaging systems and methods that provide images of biological objects in which different tissue types can be discerned.

### SUMMARY OF THE INVENTION

The present invention is directed generally to systems and methods for generating near infrared (NIR) images of biological targets, such as live subjects (human or animals). Unlike conventional infrared imaging techniques that typically rely solely on absorption of hemoglobin for generating images of blood vessels, the systems and methods of the invention utilize transmission images (images of radiation transmitted through a target via scattering by the target's tissue) at different wavelengths to obtain images of not only blood vessels but also other structures, such as bones, tendons or fascias.

In one aspect, an imaging system is disclosed that includes at least two infrared, and preferably near infrared (NIR), radiation sources for illuminating at least a portion of a biological target. The sources generate radiation at two or more different wavelengths—albeit all lying within the infrared, and preferably NIR, portion of the electromagnetic spectrum. While in some embodiments, the bandwidth of radiation generated by one source can partially overlap that of the other, in other embodiments, the sources exhibit disjoint bandwidths. The system can further comprise a detector (e.g., a CCD imaging device or any other type of low noise photodetector) that is optically coupled to the biological target so as to detect at least a portion of the illuminating radiation that is transmitted through the object (e.g., via multiple scattering by the target's tissue) to generate at least two transmission images, each corresponding to one of the illuminating wavelengths. An image processor receives the transmission images from the detector and combines them to produce a resultant image of the target. The resultant image can have a spatial resolution of about 10 μm or greater.

In a related aspect, the radiation sources can be coherent or incoherent sources generating radiation having wavelength components in a range of about 0.7 μm to about 1.1 μm, and more preferably in a range of about 0.76 μm to about 0.9 μm. For example, the sources can comprise light emitting diodes or laser diodes operating in continuous or pulse modes.

In another aspect, the image processor converts each of the images from the spatial domain to the frequency domain and scales the frequency domain images according to pre-selected scaling factors (e.g., different factors for images obtained at different wavelengths). The processor then combines the scaled frequency domain images to generate a composite frequency domain image, and converts this composite image back to the spatial domain to create the resultant image of the target. In addition, the image processor can correct the transmission images through a background illumination process prior to converting them into the frequency domain.

In a further aspect, the above imaging system further comprises a switching mechanism coupled to the radiation sources for sequentially activating them so as to illuminate the biological target with radiation at different wavelengths during different time intervals.

In yet another aspect, a filter is disposed between the biological target and the detector so as to inhibit selected wavelength components of the radiation transmitted through the target from reaching the detector, thereby improving the signal-to-noise ratio of images obtained at other wavelengths.

In other aspects, the imaging system can include a movable stage adapted for coupling to the biological target so as to allow scanning the target relative to the radiation sources and the detector for generating transmission images of different portions of the target.

The above system can be utilized to obtain infrared images of a variety of biological targets. By way of example, the system can be employed to generate images of anatomical portions of a live subject (e.g., a human subject) in which any of blood vessels, tumors, tendons, ligaments, cartilages, fascias or bones are visible.

Another aspect is directed to an infrared imaging system for illuminating an object. The system includes a source of infrared radiation generating at least two different wavelengths of radiation. For example, the radiation wavelengths can be in the range from about 0.7 μm to about 1.1 μm. One or more filters can be optically coupled to the source to allow selective illumination of the object using one of the wavelengths of source radiation, such as through the optional use of a switching mechanism to sequentially utilize the filters. A detector can be optically coupled to the object to detect at least a portion of the illuminating radiation that passes through the object. The system can also include an image processor coupled to the detector to process detector signals corresponding to at least two illumination wavelengths, and produce a resultant image from combining the detector signals. Such resultant image can be the result of converting the detector signals to frequency data corresponding to each wavelength, scaling the frequency data according to pre-selected weights, combing the weighted data to generate composite frequency data, and converting the composite frequency data into the resultant image.

In yet another aspect, a method for obtaining an NIR image of a biological target is disclosed that calls for illuminating at least a portion of the target with radiation having at least two different wavelength components. The terms “wavelength” and “wavelength component,” as used herein, can refer to a single wavelength or a wavelength band, e.g., having a bandwidth in a range of about 40 nm to about 50 nm, spanned about a single wavelength. The illumination at the two wavelengths can be achieved, for example, in two separate time intervals. At least a portion of the illuminating radiation that is transmitted through the target is detected to generate at least two transmission images, each corresponding to one of the wavelengths. The images are then combined to generate a resultant NIR image of the target.

The step of combining the images obtained at different wavelengths can comprise: converting each image from the spatial domain to the frequency domain (e.g., by applying a Fourier transformation to the image), scaling the frequency domain images based on pre-selected weights, summing the scaled images to generate a composite frequency domain image (e.g., by summing weighted Fourier coefficients of individual frequency domain images), and converting the composite frequency domain image back to the spatial domain to generate the resultant NIR image.

In a related aspect, in the above method, the transmission images are corrected via a background illumination process prior to their conversion from the spatial domain to the frequency domain.

In another aspect, a system for generating NIR images of anatomical structures of a subject is disclosed that includes a plurality of NIR radiation sources generating radiation at different wavelengths, and a detector suitable for detecting NIR radiation. The radiation sources and the detector are positioned so as to allow placement of at least a portion of the subject therebetween. The sources illuminate this portion and the detector detects at least a portion of the illuminating radiation that passes through the subject to generate transmission images, each of which corresponds to one of the wavelengths. The system further includes an image processor that is electrically coupled to the detector to collect and combine those transmission images so as to generate a resultant NIR image that shows one or more of the anatomical structures.

Further understanding of the invention can be obtained by reference to the following detailed description, in conjunction with the associated drawings, which are described briefly below.

### BRIEF DESCRIPTION OF THE DRAWINGS AND ILLUSTRATIVE IMAGES OF SOME OBJECTS

FIG. 1A schematically presents an illustrative embodiment of an imaging system according to the teachings of the invention,

FIG. 1B presents a block diagram of elements of a general illustrative embodiment of an imaging system, according to the teachings of the invention,

FIG. 2 is a diagram depicting various steps of image processing according to one embodiment of the invention,

FIG. 3 is a flow chart depicting various steps of an embodiment of a method according to the teachings of the invention for processing transmissions images of a biological target obtained at different wavelengths to generate a resultant NIR image of that target,

FIGS. 4A and 4B present NIR images of the human hand obtained by employing the teachings of the invention in which bones, tendons, blood vessels and other tissues are visible,

FIG. 5 presents illustrative NIR images of tumors injected into mice (the images were obtained by utilizing an imaging system in accordance with the teachings of the invention),

FIGS. 6A-6I present illustrative infrared and x-ray images of a frog's head,

FIG. 7A presents an image of a wrist at optically visible wavelengths,

FIG. 7B presents an infrared image of the wrist in FIG. 7A using a particular scanning mode,

FIG. 7C presents and infrared image of the wrist in FIG. 7A using a scanning mode different than used to produce FIG. 7B,

FIG. 8A presents an image of a frog's leg at optically visible wavelengths,

FIG. 8B present an infrared image of the frog's leg shown in FIG. 8A,

FIG. 9A presents an infrared image of a frog illuminated with radiation at a wavelength of about 0.72 μm,

FIG. 9B presents an infrared image of the frog of FIG. 9A illuminated with radiation at a wavelength of about 0.9 μm, and

FIG. 9C presents an infrared image created by weighting the frequency components of FIGS. 9A and 9B in a 60:40 ratio of shorter wavelength to longer wavelength image and deconvolving the composite image into the spatial domain.

### DETAILED DESCRIPTION

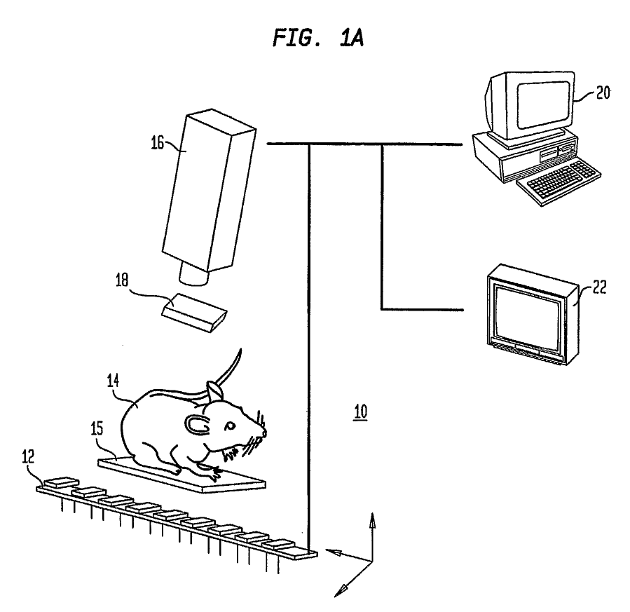

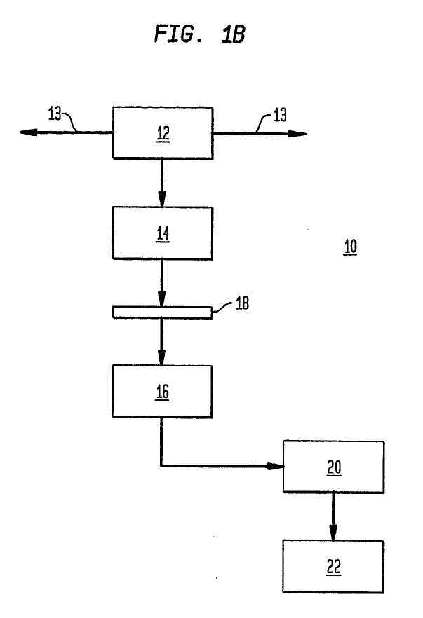

FIG. 1A schematically depicts an illustrative embodiment of an imaging system 10 according to the teachings of the invention that includes a plurality of NIR sources 12 that generate radiation for illuminating a portion, or the entirety, of a biological object—in this case shown as a small animal 14, though it can be other biological objects such as different anatomical portions of a human subject. FIG. 1B presents a block diagram depicting general elements of an imaging system 10 according to the teachings of the invention, the corresponding elements of FIG. 1A being particular examples of the general elements shown in FIG. 1B. Block 12 represents one or more NIR sources that can be optionally scanned, e.g., by a scanner 13, in at least one, and more typically in two dimensions, so as to illuminate a biological object 14 mounted on a platform 15. Such scanning can be conducted, for example, in accordance with a predefined pattern. The platform can be transparent to the radiation from the sources. In some embodiments, a plurality of narrow band NIR sources can be employed, e.g., each generating radiation with a different wavelength. In other embodiments, at least one broad source can be utilized, e.g., a light emitting diode (LED), that provides radiation having a plurality of wavelength components. In such embodiments, one or more filters, represented herein by block 18, can be used to selectively transmit radiation having a particular wavelength to the object 14. Block 16 represents a detector for detecting at least a portion of the radiation transmitted through the object, and block 20 represents an image processing system that receives detection signals from the detector to generate an image of the object in accordance with the teachings of the invention, as discussed further below. The image can be displayed on a display 22.

In general, NIR sources generate radiation in the near infrared portion of the electromagnetic spectrum (e.g., in a range of about 0.7 μm to about 1.1 μm, and preferably in a range of 0.76 μm to about 0.9 μm) and can exhibit disjoint or partially overlapping bandwidths (i.e., any two sources having at least one differing wavelength component). In the illustrative embodiment of FIG. 1A, each of a plurality of sources generates radiation with a different wavelength. Although each source is depicted herein as a single radiation unit, in some embodiments, it can itself be composed of an array of radiation generators with a common wavelength. For example, a radiation source can comprise an array of light-emitting diodes, each diode generating radiation within a selected NIR bandwidth. In one example, 66 narrow-band NIR emitters, obtained from Sacher Lasertechnik GmbH (Germany), can be utilized as the source with each emitter generating radiation in a chosen narrow bandwidth range of about 40 to about 50 nanometers for every selected wavelength in a range from about 0.72 μm to about 1 μm. In another embodiment, the source can be a broadband emitter that can operate in conjunction with a set of filters as discussed further below.

At least a portion of the radiation transmitted through the illuminated object, for example, via multiple scattering events within the object, is detected by a detector 16. As the scattering of radiation within the object is generally a noisy process, in many embodiments, the detector is selected to have a super high sensitivity (low noise) to provide an acceptable signal-to-noise ratio. A commercially available NIR detector marketed by Security Systems, Co. under trade designation model BKC-1 is an example of a detector suitable for use in the practice of the invention.

In some embodiments, a detector can also be embodied as a video camera or other devices capable of processing received radiation in a manner to create a moving picture image. For example, such a detector can process images at a frame rate of about one frame per 30 milliseconds. A video camera can be coupled with an appropriate image processor and display to create real-time NIR video images of a subject in accordance with the teachings of the invention. For example, a small low noise video camera commercially available from Supercircuits Ltd. can be used for this purpose. Accordingly, in some embodiments of the invention, a video camera can allow real-time imaging in which the movements of a biological object (e.g., the movements of the fingers of a hand) can be captured in the NIR in real-time, thereby providing a richer set of information that a user (e.g., a medical practitioner) can use to study a particular subject, e.g., to diagnose a medical condition. This provides distinct advantages over the use of x-rays for studying a subject, as obtaining images of a moving object by conventional x-ray imaging can be impractical, e.g., due to diffraction effects.

In the illustrative embodiment of FIG. 1A, an NIR band pass filter 18 is disposed in front of the detector 16 so as to lower, and preferably eliminate, detection of radiation at wavelengths that lie outside the desired bandwidths, thus improving the signal-to-noise ratio. Suitable NIR filters are commercially available.

A series of images of the illuminated portion of the object at different NIR wavelengths corresponding to those generated by the radiation sources can be obtained by selectively activating the sources, e.g., sequentially, and detecting the radiation transmitted through the object by the detector 16. By way of example, such images can be obtained by scanning the one or more sources, e.g., in a chosen direction 13 as depicted in FIG. 1B, so to illuminate the biological object in a predetermined pattern. For example, the scanner shown schematically in FIG. 1B can be a stepper motor to which the radiation source(s) are coupled. The stepper motor can be configured to move the source(s) relative to the biological object in a designated pattern. For example, in some embodiments, the movement of the sources can sequentially expose the object to different radiation wavelengths, with the detector generating transmission images at those wavelengths. Such images can be combined, e.g., in a manner discussed in more detail below, to generate a resultant NIR image of the object.

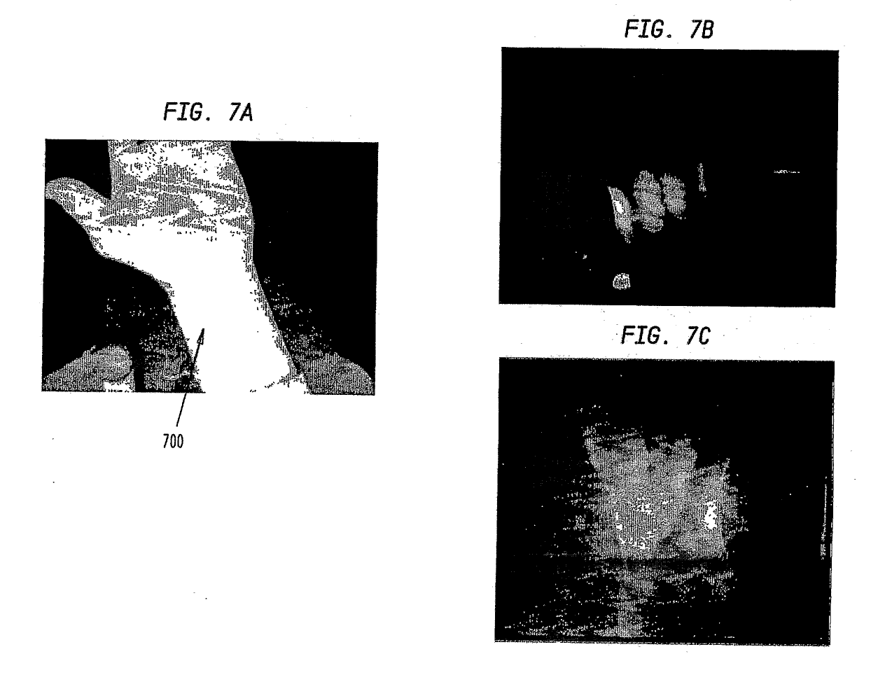

Different scanning modes can be utilized with the NIR radiation sources to provide images that can emphasize particular qualities. Examples of such scanning modes are described with reference to the prints of FIGS. 7A-7C. A section of a human wrist 700, shown in FIG. 7A, is scanned in two modes. In one mode, the scanning is performed to emphasize particular bone and blood vessel features, as shown in FIG. 7B. In this mode, the entire wrist area was illuminated by two wavelengths of radiation, about 0.72 μm and about 0.9 μm. The images were converted into respective frequency domain sets, combined using a weighting factor of 60:40 for the about 0.72 μm and about 0.9 μm sets, respectively, and the composite was deconvoluted to produce the image of FIG. 7B; this process is described in further detail herein. In another mode, the scanning is performed to emphasize small-scale external features such as hairs on the skin of the scanned patient as shown in FIG. 7C. For this mode, the source is scanned across the object using a wavelength of about 0.9 μm. Given the fine details of hair thickness that can be imaged with this technique, it is clear that some embodiments of the invention are directed to imaging systems, as described herein, that can provide high resolution IR images (e.g., spatial detail as small as about 10 μm to about 15 μm) without the use of a detector that restricts the view to a very small window size, i.e., a wide view detector can be utilized. In other words, a high resolution image of a portion, or an entire, of a biological object can be obtained while concurrently collecting image data from that portion by a wide-area detector.

Though scanning is described above with respect to moving one or more sources of radiation, it is understood that scanning utilizes relative movement between the biological target and the radiation source. Accordingly, such relative movement can also be achieved by moving the target using a stage or other devices, or even employing a series of devices to move both the source and the target. Further, in some embodiments, such devices can be coupled to provide coordinated movement.

As noted above, in some embodiments, one or more filters can also be used in conjunction with a broadband emitting source to illuminate an object with a plurality of different NIR wavelengths. For example, a broadband LED can be used as a source for emitting a range of NIR wavelengths. For each image in a collection of images obtained at different wavelengths, the radiation from the LED can pass through a suitable filter that selects the radiation wavelength desired for that image. In this manner, each filter can provide a specific narrow bandwidth, resulting in a transmission image through an object of interest at the wavelength associated with the filter.

An image processor 20, e.g., a computer supplied with the requisite image processing software in accordance with the teachings of the invention, collects and processes the received images in a manner discussed below to generate a resultant NIR image that can show not only blood vessels but also tendons, muscles, ligaments, cartilages and bones.

In the illustrative embodiment of FIG. 1A, a real-time display 22 is also provided for real-time viewing of the NIR images collected by the detector 16. Although in this embodiment, focusing systems (e.g., one or more convergent lenses) are not employed for focusing radiation from the sources onto the biological object, in other embodiments, such focusing systems can be incorporated into the imaging system 10. Further, although not shown in FIG. 1A, the detector 16 can include one or more lenses for focusing the radiation transmitted through the object onto its image-forming elements (e.g., photodetectors ). In other embodiments, an NIR imaging system, with features described herein, can receive detector signals from illuminated radiation, and utilize an image processor to process the detector signals. Display of any images (e.g., component or combined data images) from the system can be obtained after signal processing, which need not be during real-time operation of the illuminating radiation.

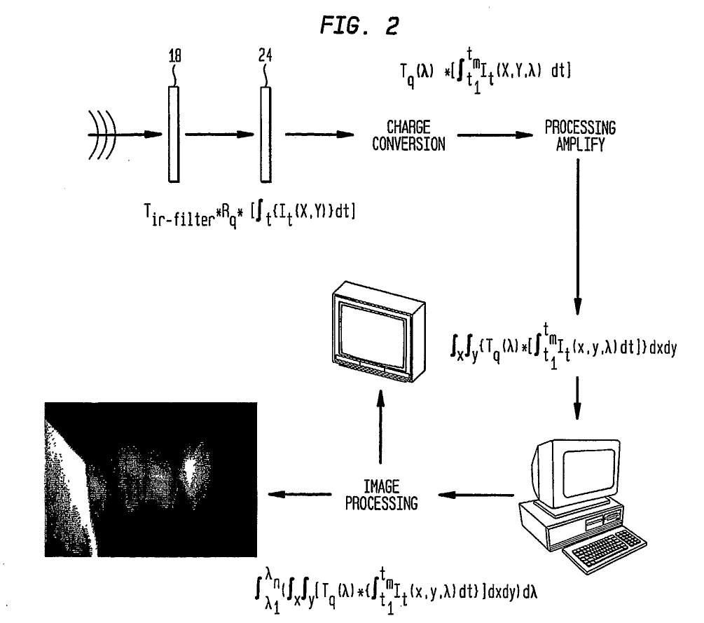

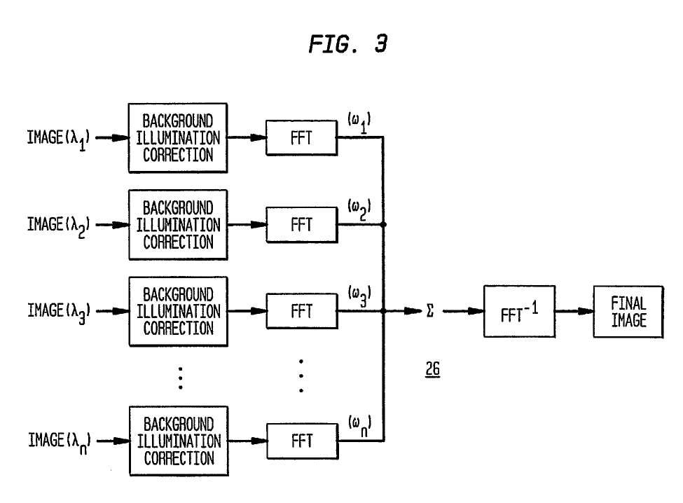

The various stages of image collection and processing utilized in many embodiments of the invention can be better understood by reference to FIG. 2. Each portion of the biological object illuminated by the NIR radiation can function as a scattering source to emanate radiation at a given intensity towards the infrared filter 18. The infrared filter can be characterized by a wavelength-dependent transmission coefficient, designated here by T_{ir-filter}, that scales the intensity of the radiation passing through it. The filter is preferably selected to exhibit a high and substantially flat transmission coefficient over the wavelength range of interest, which coefficient decreases precipitously for wavelengths beyond that range. Such filters include commercially available filters produced by Edmund Scientific, such as the Tech Spec™ Shortpass Filters that have an average transmission of at least about 85% and sharp transition cut-offs. A shutter 24 exposes the detector to the radiation transmitted through the filter 18 for a selected time duration, herein designated as a duration between a time (t₁) to a time (tₘ), and the detector converts the optical energy incident thereon into electrical energy (T_{q}(λ) is transmission coefficient). Thus, for each illumination wavelength (λ) and each cross-sectional location of the object (x,y), the detector's output corresponding to the intensity of light emanating from that location in response to illumination can be designated as T_{q}(λ)*[∫ₜ₁^{tm}Iₜ(x,y,λ)dt].

Similar output signals can be obtained for other locations of the biological object at each wavelength of interest (e.g., each wavelength corresponding to radiation generated by one of the sources) and combined to generate an image at that wavelength. A plurality of such images at different wavelengths can then be combined, e.g., in a manner discussed below, to obtain a resultant image.

More particularly, as shown in a flow chart 26 of FIG. 3, in the illustrative embodiment of FIGS. 1A and 1B, the transmission images obtained at different wavelengths are initially corrected through a background illumination process. Such a process can help enhance the contrast of an image, which may be overly bright or dim. In one embodiment, the images can be collected serially while maintaining the examined object stationary as the sources are sequentially activated. As such, no image alignment is necessary. In other embodiments, however, image alignment may be needed. Background illumination correction can be applied to recalibrate the average brightness of an image to adjust for situations with too much, or too little, brightness. By way of example, in some instances in which the intensity of each of the pixels of an image is characterized by a grayscale value (e.g., a value between 0 and 256, the latter being white and the former being black), an average value of all pixels can be calculated. If the average value exceeds a predefined threshold, it can be subtracted from each of the pixels to produce a recalibrated image. Alternatively, the average value can be multiplied by some constant before being subtracted from the pixel values. In other cases, instead of or in addition to considering the image brightness, the image contrast, e.g., characterized by a maximum or a minimum contrast defined, e.g., by the maximum or minimum difference between grayscale pixel values, can be considered. Such recalibration of the images can be obtained, e.g., by an image processing software provided in the image processor 20.

After undergoing background illumination correction, the images can be converted to frequency spatial domain. Conversion of imaging data from the spatial domain to the frequency domain can be accomplished using a variety of conventional techniques such as Fourier Transforms (e.g., by using a Fast Fourier Transform technique (FFT) or a conventional Fourier Transform technique). Subsequently, corresponding elements can be weighted and then combined, e.g., by summing the weighted Fourier coefficients of the frequency domain images. The summed Fourier coefficients can be also normalized to suppress noise as well as uncorrelated signals. The final Fourier spatial image can then be inverted back to the spatial domain to generate the resultant image. The choice of weighting factors for the frequency domain images can depend upon a number of resultant image merit factors that can include contrast, brightness, and/or sharpness. In many embodiments, the selection of the weighting factors can also depend upon the characteristics of the object being imaged. In general, for thicker biological object cross sections and/or for objects having higher densities (and more generally for objects characterized by a higher degree of radiation scattering) images obtained at shorter wavelengths (i.e., higher frequencies) are weighted more heavily than those obtained at longer wavelengths. In one example, a default value of 60% is assigned to the weighting factor for a shorter wavelength component, and a value of 40% is assigned to the weighting factor for a longer wavelength components. If the quality of the resultant image is not satisfactory (e.g., as determined by a human operator or an image processing algorithm), the default values can be changed (e.g., 70:30 or 50:50 wavelength weighting ratios) until a desired resultant image is obtained.

The systems and methods of the invention for obtaining NIR images of biological objects can be advantageously employed in a variety of biomedical applications. For example, they can be utilized to visualize blood vessels. Unlike conventional techniques for visualizing blood vessels, the systems and methods of the invention allow obtaining images of blood vessels at much greater depths. This characteristic allows the use of the systems and methods of the invention in high precision surgery (e.g., infrared images would warn a surgeon whose knife may be approaching dangerous areas). In particular, since blood vessels are normally accompanied by nerves, they can be used as surgical guides, e.g., to avoid severing of main neurovascular bundles during surgery. Further, blood vessels can be utilized as landmarks of critical organs. The visualization of blood vessels also allows locating main vessels for drawing blood from or inserting endoscopic tools into a patient. Moreover, advanced understanding of correlations between medical conditions and blood content allows the use of imaging of the blood vessels as a universal diagnostic tool. For example, since proliferation of blood vessels can be used as an indicator of the progress of tissue regeneration, reattachment of severed limbs, healing processes, among others, the techniques of the invention can also provide diagnostic tools for such purposes. By allowing visualization of blood vessels, the methods and the systems of the invention provide diagnostic tools for assessing the severity of tissue damage from concussion, swelling and bleeding, as well as for identifying tumor growth, which is usually associated with formation of abnormal blood vessels.

In addition, the NIR imaging methods and systems of the invention allow obtaining NIR video images of moving objects, as discussed above. This can be particularly advantageous, e.g., for diagnostics purposes. For example, a medical professional can view an NIR video image of a patient's hand as the patient closes and opens her hand.

The applications of the teachings of the invention are not, however, limited to visualization of blood vessels. In particular, unlike conventional infrared imaging techniques, they can be employed to identify other soft tissue types, such as tumors, as well as ligaments, cartilages, tendons, fascias, bones, or even deep organs, e.g., by varying the angle of transmitted sources and detectors. Therefore, the teachings of the invention open new avenues in mammography diagnostics, in-situ alignment of broken bonds, observation of arthritis and change of bone dimensions (osteoporosis), etc.

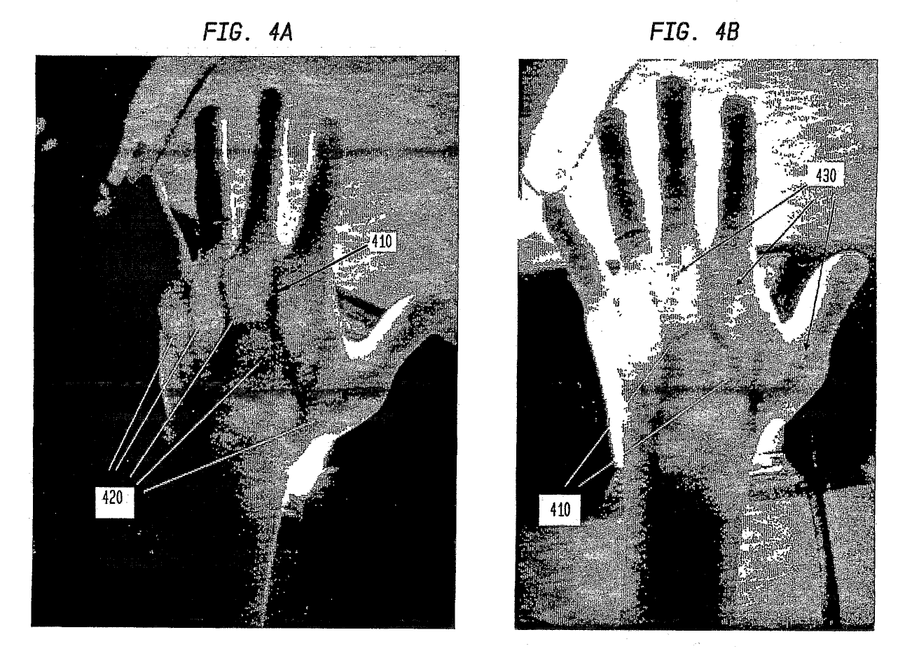

To show the efficacy of the teachings of the invention for obtaining NIR images of biological targets and only for illustration purposes, various NIR images of different biological targets are presented. FIGS. 4A and 4B present NIR images of a human hand obtained by utilizing a system of the invention. In these images, not only blood vessels but also bones, tendons and other tissues are visible. FIGS. 4A and 4B were obtained by combining infrared images at about 0.68 μm and at about 0.97 μm. The individual images were converted to the frequency domain and combined using weighting factors of 60:40 for the shorter to longer wavelength data, respectively. The combined frequency domain image data was inverted to the spatial domain to produce the images of FIGS. 4A and 4B. The differences in contrast were obtained by utilizing background illumination correction to represent different average intensities for each of the images, FIG. 4B having the higher average intensity.

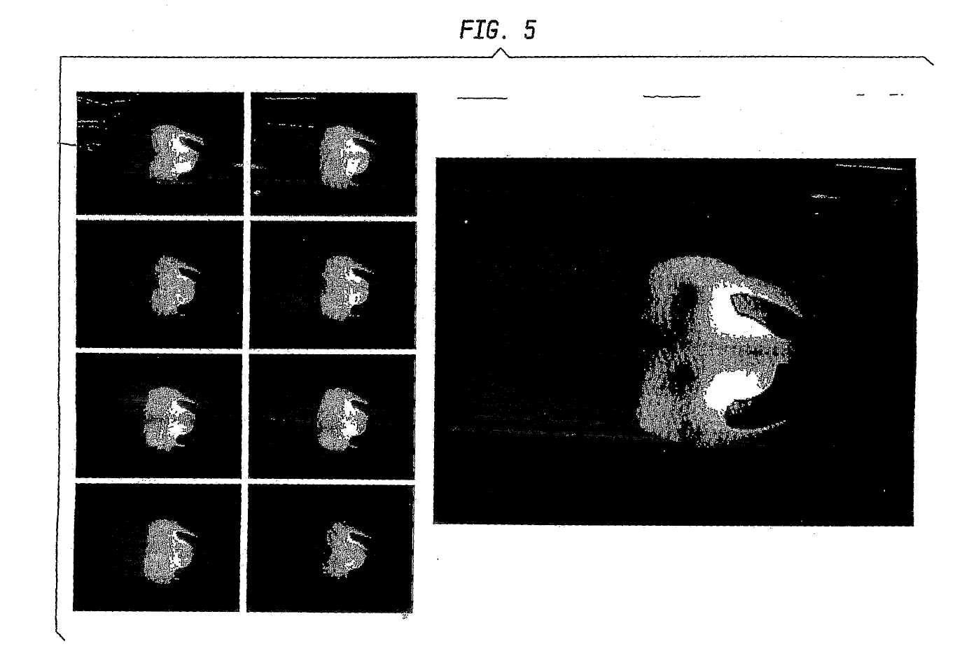

FIG. 5 presents NIR images of tumors injected into mice. Each of the images is formed by combining two infrared images at about 0.68 μm and at about 0.97 μm using weighting factors of 60:40, respectively. The images differ in the amount of background illumination correction that is applied to each. The two dark oval spots visible in these images around tail region, on both sides of the spine, are the sites of neuroblastoma cells injection, visualized 17 to 24 hours after the injection.

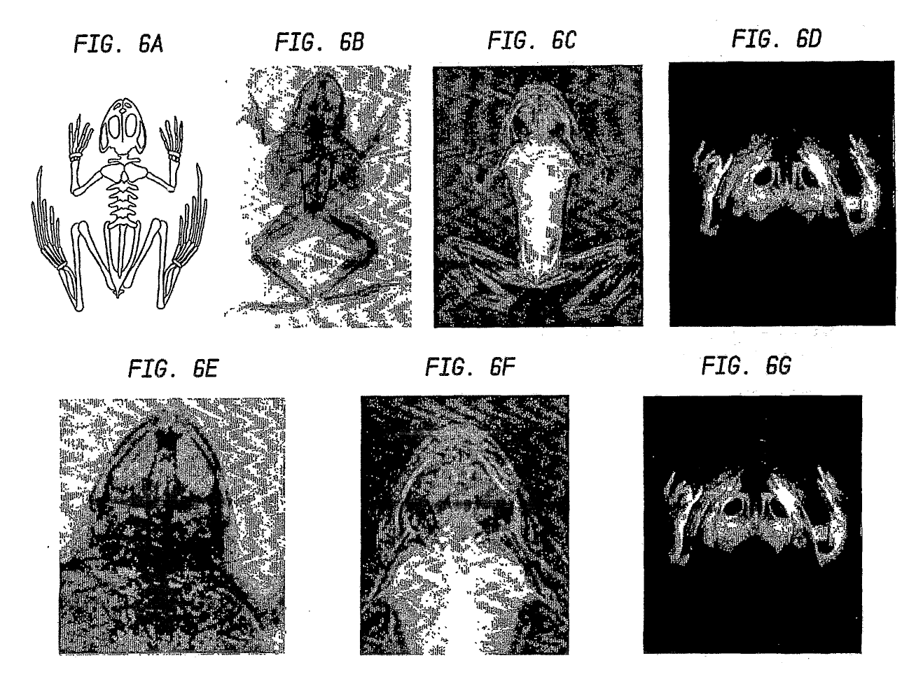

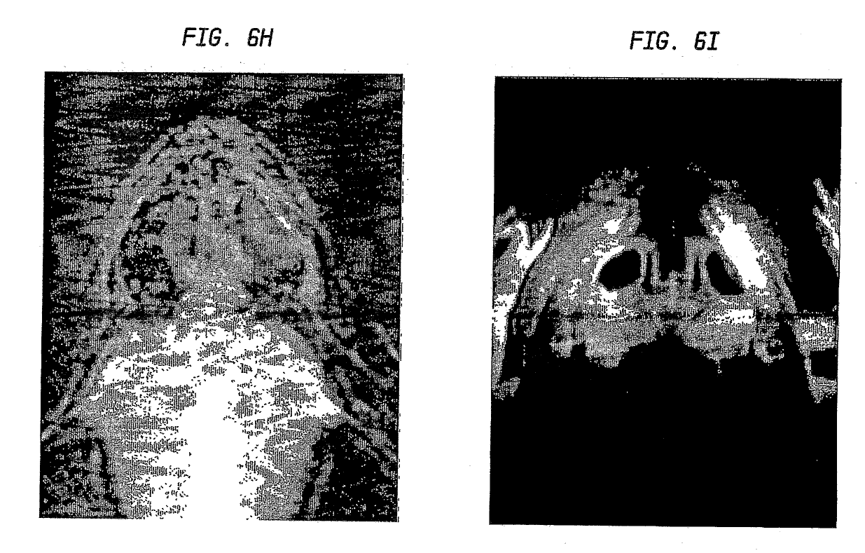

FIGS. 6A-6G provide a comparison of infrared images of a frog's head obtained by utilizing an illustrative system according to the invention with x-ray images of the frog's head. FIG. 6A provides a reference for the frog's skeletal system. FIG. 6B provides a conventional x-ray image, with an enhanced processing image shown in FIG. 6C. FIG. 6D provides an NIR image created using the techniques described in the present application. FIGS. 6E, 6F, and 6G are enlarged head sections of FIGS. 6B, 6C, and 6D, respectively. While the bones of the scalp are visible in the infrared and x-ray images, the infrared images allow, in addition, visualization of the brain and eyes tissues, which present a great deal of important details. FIGS. 6H and 6I present enlarged frames of FIGS. 6F and 6G, respectively, and also scale the images such that the frog head size is similar in both images. Accordingly, a more accurate comparison of the x-ray and NIR images can made by looking at FIGS. 6H and 6I.

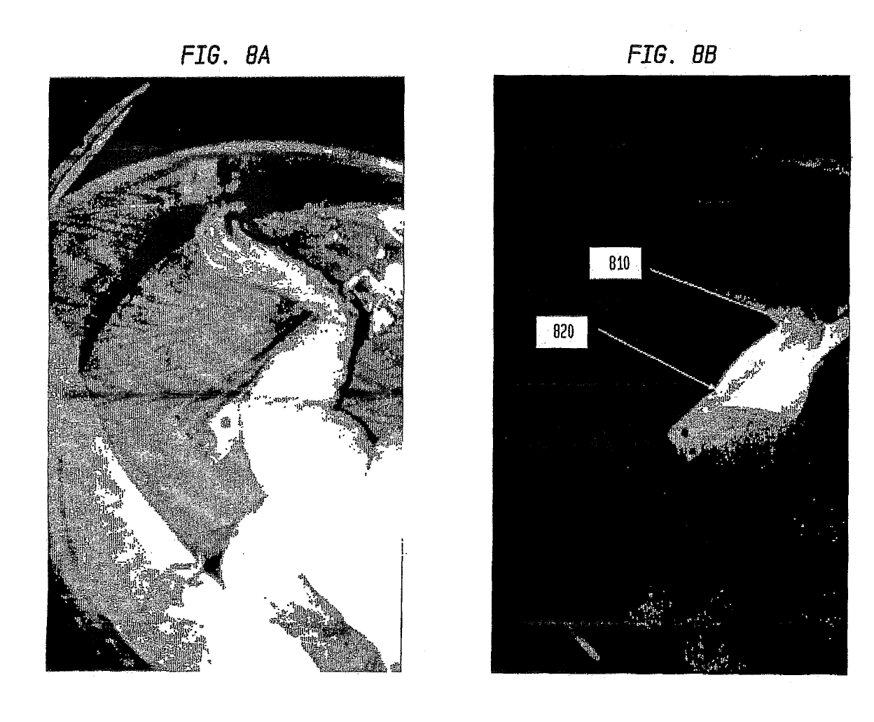

FIGS. 8A and 8B provide a visible optical image of a frog leg and a NIR image of the same leg, respectively. The NIR image of FIG. 8B shows the tibia 820 and ankle joint 810, exemplifying the multiple types of tissue that can be imaged by embodiments of the invention.

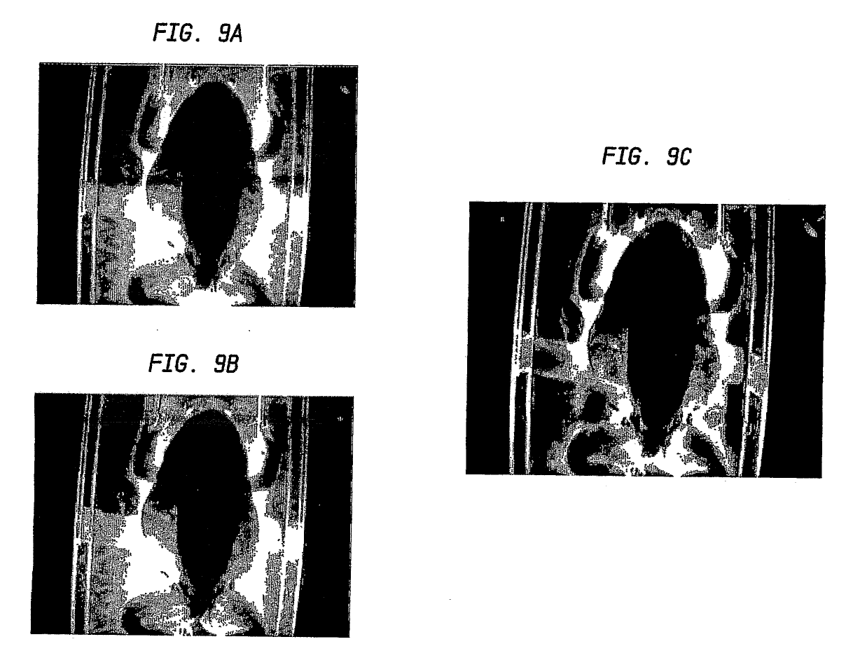

FIGS. 9A-9C provide images corresponding to an embodiment of combining imagery data from different imaging wavelengths. FIGS. 9A and 9B infrared images of a frog formed from illumination using wavelengths of about 0.72 μm and about 0.9 μm, respectively. FIG. 9C represents an image formed from combining the Fourier components of FIGS. 9A and 9B using a relative weighting factor of 60 to 40 in terms of the shorter wavelength component to longer wavelength component and inverting the combination to the spatial domain. FIG. 9C is a clearer image that shows better tissue details then either of the other infrared images.

It should, however, be understood that these images are provided only for illustrative purposes and are not intended to necessarily indicate optimal image qualities that can obtained by practicing the teachings of the invention.

As discussed above, both narrow-band and broad-band sources can be utilized in the infrared imaging systems according to the teachings of the invention. When utilizing a broadband source, it can be coupled to a plurality of filters to generate radiation at multiple wavelengths for illuminating a target. For example, different filters, each allowing passage of a selected wavelength band, can be sequentially coupled to the source to obtain illuminating radiation at several wavelengths.

Those having ordinary skill in the art will appreciate that various changes can be made to the above embodiments without departing from the scope of the invention.

What is claimed is:

1. An imaging system, comprising
at least two infrared radiation sources adapted for illuminating at least a portion of a biological target, said sources generating radiation at two different wavelengths,
a detector optically coupled to the biological target so as to detect at least a portion of the radiation from the sources that is transmitted through said illuminated portion of the target,
an image processor coupled to the detector for collecting at least two transmission images each corresponding to one of said different wavelengths,
wherein said image processor combines said images to obtain a resultant infrared image of the target.

2. The imaging system of claim 1, wherein said sources generate radiation having wavelength components in a range of about 0.7 μm to about 1.1 μm.

3. The imaging system of claim 2, wherein the radiation sources operate in any of a continuous or a pulsed mode.

4. The imaging system of claim 1, wherein said image processor converts each of said images into the frequency domain, scales the frequency domain images according to pre-selected weights, combines said weighted images to generate a composite frequency domain image, and converts said composite image to a resultant spatial domain image.

5. The system of claim 1, further comprising a switching mechanism coupled to said sources for sequentially activating the sources so as to illumine the biological target in different time intervals.

6. The system of claim 1, further comprising a filter positioned between said biological target and the detector so as to inhibit selected wavelength components of the radiation transmitted through the target from reaching the detector.

7. The system of claim 1, further comprising at least one focusing element coupled to said radiation sources for focusing radiation from said sources onto the target.

8. The system of claim 1, further comprising at least one focusing element for focusing said transmitted radiation onto the detector.

9. The system of claim 1, wherein said detector comprises a low noise detector.

10. The system of claim 1, wherein said detector comprises a CCD imaging device.

11. The system of claim 1, wherein at least one of said sources generates coherent radiation having wavelength components within the near infrared (NIR) portion of the electromagnetic spectrum.

12. The system of claim 1, wherein at least one of said sources generates incoherent radiation having wavelength components within the near infrared (NIR) portion of the electromagnetic spectrum.

13. The system of claim 1, further comprising a movable stage adapted for coupling to the biological target to allow scanning the target relative to the radiation sources and the detector so as to generate transmission images of different portions of the target.

14. The system of claim 1, wherein said biological target comprises an anatomical portion of a live subject and said image processor generates an image exhibiting any of blood vessels, tendons, fascias, ligaments, tumors, cartilages or bones in said anatomical portion.

15. The system of claim 1, wherein said resultant image exhibits a spatial resolution of about 10 μm or greater.

16. An infrared imaging system, comprising
a source of infrared radiation generating at least two different wavelengths, said source being adapted for illuminating at least a portion of an object,
one or more filters optically coupled to the source to allow selectively illuminating said object with radiation from said source having one of said wavelengths,
a detector optically coupled to the object so as to detect at least a portion of the illuminating radiation passing through the object,
an image processor coupled to the detector for processing detector signals corresponding to at least two illumination wavelengths, and generating a resultant image of the object by combining said detector signals.

17. The infrared imaging system of claim 16, wherein the source of infrared radiation is configured to generate infrared radiation having at least one wavelength component in a range of about 0.7 μm to about 1.1 μm.

18. The infrared imaging system of claim 16, wherein the image processor is configured to convert detector signals corresponding to each of the at least two illumination wavelengths into the frequency data, scale the frequency data for each illumination wavelength according to pre-selected weights, combine weighted data to generate composite frequency data, and convert said composite frequency data into a resultant spatial domain image.

19. The infrared imaging system of claim 16, further comprising a switching mechanism coupled to the source of infrared radiation for sequentially utilizing the one or more filters to selectively illuminate the object.

20. A method for obtaining a near infrared (NIR) image of a biological target, comprising
illuminating at least a portion of the biological target with radiation having at least two different wavelength components,
detecting at least a portion of said illuminating radiation transmitted through the target to generate at least two transmission images each corresponding to one of said wavelength components, and
combining said two images to generate a resultant NIR image of the target.

21. The method of claim 20, wherein said combining step further comprises:
converting each image from the spatial domain to the frequency domain,
scaling the frequency domain images based on pre-selected weights,
combining said scaled images to generate a composite frequency domain image, and
converting said composite frequency domain image to said resultant NIR image.

22. The method of claim 21, further comprising correcting said transmission images through a background illumination process prior to said step of converting the images from the spatial domain to the frequency domain.

23. The method of claim 21, further comprising selecting said illuminating radiation to have wavelength components in the near infrared (NIR) portion of the electromagnetic spectrum.

24. The method of claim 21, wherein said step of converting images from the spatial domain to the frequency domain comprises applying a Fourier transformation to said images.

25. The method of claim 24, wherein said combing step comprises summing weighted Fourier coefficients of the frequency domain images.

26. A system for generating a near infrared (NIR) image of anatomical structures of a subject, comprising:
a plurality of near infrared (NIR) radiation sources generating radiation at different wavelengths,
a detector suitable for detecting NIR radiation,
said sources and the detector being positioned so as to allow placement of at least a portion of the subject therebetween for illumination by said sources, said detector detecting at least a portion of the illuminating radiation passing through said subject to generate transmission images each corresponding to one of the wavelengths,
an image processor electrically coupled to said detector to collect and combine said transmission images to generate a resultant NIR image showing one or more of said anatomical structures.

27. The system of claim 26, further comprising a switch coupled to the sources for activating thereof so as to illuminate the subject portion with radiation at said different wavelengths.

28. The system of claim 27, wherein said switch sequentially activates the sources while said subjects remains stationary relative to the sources and the detector.

29. The system of claim 27, wherein said image processor employs said transmission images obtained at different wavelengths so as to provide visualization of any of different tissue types, bones, ligaments, blood vessels, and cartilages.
